(19) United States
(12) Patent Application Publication
(10) Pub. No.: US 2022/0296176 A1
(43) Pub. Date: Sep. 22, 2022

(54) PROCESSING BIOLOGICAL DATA

(71) Applicant: PREVENTICUS GMBH, Jena (DE)

(72) Inventor: Thomas Hübner, Jena (DE)

(21) Appl. No.: 17/708,087

(22) Filed: Mar. 30, 2022

Related U.S. Application Data
(62) Division of application No. 15/744,694, filed on Jan. 12, 2018.

(30) Foreign Application Priority Data
Jul. 16, 2015 (EP) 15177174.8
May 17, 2016 (EP) 16170035.6

(51) Int. Cl. A61B 5/00; A61B 5/0205; A61B 5/021; A61B 5/024; A61B 5/08; G16H 50/20

(52) U.S. Cl. CPC A61B 5/7278; A61B 5/7282; A61B 5/0205; A61B 5/02108; A61B 5/02405; A61B 5/0816; G16H 50/20; A61B 5/6898; A61B 5/7246; A61B 5/02416; A61B 5/02438

(57) ABSTRACT

An apparatus for determining data pertaining to the heart rate, respiratory rate, and/or blood pressure of a human subject based on pulse waveform analysis is disclosed. The apparatus comprises a control unit and a sensor unit configured for providing pulse wave data representative of a heartbeat of a subject. The control unit is configured for receiving the pulse wave data, and selecting a portion of the pulse wave data indicative of a plurality of heart periods. Based on the portion of the pulse wave data indicative of a plurality of heart periods, the control unit is further configured for determining a blood pressure variability; determining a respiratory rate variability; and determining a heart rate variability. Based on at least two of the blood pressure variability, the respiratory rate variability, and the heart rate variability, a correlation value may be determined from which a medical condition of the subject is determined.

### CROSS-REFERENCE TO RELATED APPLICATIONS

This application is a divisional of U.S. application Ser. No. 15/744,694, filed on Jan. 12, 2018, which is a U.S. National Phase Application under 35 U.S.C. § 371 of International Application No. PCT/EP2016/066944, filed on Jul. 15, 2016, which claims priority to European Application No. 15177174.8, filed Jul. 16, 2015 and European Application No. 16170035.6, filed May 17, 2016. This disclosures of the prior applications are considered part of (and incorporated by reference in) the disclosure of this application.

### TECHNICAL FIELD

The present invention relates to processing of biological data. Based on pulse waveform analysis, data pertaining to, for example, the heart rate, respiratory rate, and/or blood pressure of a human subject are determined and processed.

### BACKGROUND ART

The primary causes for diseases such as heart attack and stroke are conditions that are often hard to detect and do not entail pronounced symptoms. For example, hypertension and coronary artery disease (CAD) are among the primary causes for heart attack and atrial fibrillation (AFIB) is one of the primary causes for stroke. Regular measurement of, for example, blood pressure, heart rate, respiratory rate and a detailed analysis of such biological parameters of a subject can be employed in detecting hypertension, AFIB, CAD, and other conditions or the early onset thereof. However, these measures are often not employed on a regular basis.

AF is the most common arrhythmia encountered in clinical practice and its paroxysmal nature renders its detection difficult. Without specific therapy, the risk for stroke and congestive heart failure increases significantly. The paroxysmal nature of AF may be present for years before it becomes persistent. This particular property of AF renders its detection difficult and often unsuccessful. Recent trials (see, e.g., Gladstone D J, Spring M, Dorian P, Panzov V, Thorpe K E, Hall J, et al. “Atrial fibrillation in patients with cryptogenic stroke”, The New England journal of medicine 2014; 370: 2467-2477; Sanna T, Diener H C, Passman R S, Di Lazzaro V, Bernstein R A, Morillo C A, et al. “Cryptogenic stroke and underlying atrial fibrillation”, N Engl J Med 2014; 370: 2478-2486) support the use of intensified diagnostic strategies to detect AF in selected patients, although the employed methods can be costly or inconvenient. Even with the rapidly increasing knowledge in this field, the relevance of subclinical AF and the temporal correlation between AF and stroke remains controversial and is still being addressed in ongoing trials (see, e.g., Healey J S, Connolly S J, Gold M R, Israel C W, Van Gelder I C, Capucci A, et al. “Subclinical atrial fibrillation and the risk of stroke”, N Engl J Med 2012; 366: 120-129; Ziegler P D, Glotzer T V, Daoud E G, Wyse D G, Singer D E, Ezekowitz M D, et al. “Incidence of newly detected atrial arrhythmias via implantable devices in patients with a history of thromboembolic events”, Stroke; 41: 256-260).

The use of smartphones and smart watches in medical practice has received increased attention in the recent past. Suitable devices are equipped with plethysmographic sensors configured to monitor the heart rate. Pulse wave analysis can be employed in order to record and process different biological properties of a patient, based on which certain medical conditions can be determined.

Blood pressure is the pressure exerted by circulating blood upon the walls of blood vessels and is one of the principal vital signs of a person. It is regulated by the nervous and endocrine systems and varies depending on a number of factors including current activity and general health condition of a person. Pathologically low blood pressure is referred to as hypotension, and pathologically high blood pressure is referred to as hypertension. Both pathologies can have different causes and can range from mild to severe, with both acute and chronic forms. Chronic hypertension is a risk factor for many complications, including peripheral vascular disease, heart attack, and stroke. Both hypertension and hypotension are often undetected for longer periods of time because of infrequent monitoring.

Hypertension is generally more common and constitutes the predominant risk factor for a cardiovascular disease and associated health problems including death, higher than those for smoking and diabetes. One major problem with hypertension is that high blood pressure does not necessarily entail pronounced symptoms and that, consequently, there are many people living their lives without realizing that they have elevated or high blood pressure. Measuring and monitoring blood pressure can be done in a number of ways, including at home, as an outpatient, or as an inpatient. However, sporadic and/or infrequent measurements are typically not meaningful enough for effective early detection of hypertension and associated diseases, due to the intervals between measurements often being too long and the measurements being done not often enough.

Medical professionals commonly measure arterial pressure using a sphygmomanometer, which historically used the height of a column of mercury to reflect the circulating pressure, and blood pressure values are typically reported in millimeters of mercury (mm Hg). For each heartbeat, blood pressure varies between systolic and diastolic pressures. Systolic pressure is the peak pressure in the arteries, occurring near the end of a cardiac cycle when the ventricles are contracting. Diastolic pressure is the minimum pressure in the arteries, occurring near the beginning of a cardiac cycle when the ventricles are filled with blood. Typical normal measured values for a resting and healthy adult are 120 mm Hg systolic pressure and 80 mm Hg diastolic pressure (i.e. 120/80 mm Hg).

Systolic and diastolic arterial blood pressures are not static but undergo natural variations from one heartbeat to the next and throughout the day (in a circadian rhythm). Variations occur in response to stress or exercise, changes in nutrition, and disease or associated medication. Blood pressure is one of the four main vital signs, further including body temperature, respiratory rate, and pulse rate, that are routinely monitored by medical professionals and healthcare providers.

Blood pressure can be measured in a noninvasive manner, including palpation, auscultatory or oscillometric methods, continuous noninvasive techniques (CNAP), and based on the pulse wave velocity (PWV) principle. Measuring blood pressure invasively, for example using intravascular cannulae, can produce very accurate measurements, but is much less common due to its invasive nature and is typically restricted to inpatient treatment.

Blood pressure in humans is significantly affected by the elasticity of the vascular system. The elasticity of the vascular system of a person depends on different factors including age, but also on the presence or absence of particular diseases or illnesses. If, for example, the elasticity of the vascular system of a patient decreases due to old age or due to the patient suffering from arteriosclerosis, the blood pressure of the patient increases.

The heart rate (HR) of a subject and the respiratory rate (RR) of a subject can also be determined by a physician using known methods for inpatient treatment. Also these measurements are typically taken only at irregular intervals and/or with long periods of time without measurements in between.

The variability of certain biological parameters, such as heart rate, respiration, blood pressure, can serve as an indicator for medical conditions, for example sleep apnea, depression, AF (or AFIB), CAD. It is noted that the term variability can mean a single variability value or measure or a plurality of values indicative of the variability of the respective parameter. Any known representations of variabilities are accepted within the scope of the present documents.

A. Seeck, W. Rademacher, C. Fischer, J. Haueisen, R. Surber, A. Voss, “Prediction of atrial fibrillation recurrence after cardioversion—Interaction analysis of cardiac autonomic regulation” have found in a study that the assessment of the autonomic regulation by analyzing the coupling of heart rate and systolic blood pressure provides a potential tool for the prediction of arterial fibrillation recurrence after CV and could aid in the adjustment of therapeutic options for patients with arterial fibrillation.

W. Poppe et al., “Eignen sich die Hüllungskurven von Arterienpulswellen für eine Fernbeurteilung psychotischer Krankheitsverläufe?”, have found that the envelope of the arterial pulse wave can be indicative of a subject being classified with respect to a particular psychosis and further indicative of a likely progression of a psychosis. This research applies to, for example, the correlation of depression with pulse wave data.

An aim of the present invention is to provide an apparatus for accurately determining biological parameters of a subject, for example heart rate, respiration, blood pressure, and the variabilities thereof, in a noninvasive manner, easily, and efficiently. It is a further aim to provide an apparatus for determining the biological parameters of a subject and the variabilities thereof with an improved accuracy.

A further aim of the present invention is to provide an apparatus for performing the non-invasive method for determining the blood pressure of a human subject. In particular, the apparatus is a mobile device, and preferably a conventional smart phone provided with a light source and an optical sensor.

### SUMMARY OF INVENTION

According to the invention, in a 1^{st} aspect there is provided an apparatus for determining a medical condition of a human subject, the apparatus comprising a control unit; and a means for providing pulse wave data representative of a heart beat of the human subject; wherein the control unit is configured to perform the steps of: receiving the pulse wave data; selecting a portion of the pulse wave data indicative of a plurality of heart periods; for the portion of the pulse wave data indicative of a plurality of heart periods: —determining a blood pressure variability based on the pulse wave data of the portion of the pulse wave data indicative of a plurality of heart periods; —determining a respiratory rate variability based on the pulse wave data of the portion of the pulse wave data indicative of a plurality of heart periods; and —determining a heart rate variability based on the pulse wave data of the portion of the pulse wave data indicative of a plurality of heart periods; and determining at least one correlation value based on at least one of the blood pressure variability, the respiratory rate variability, the heart rate variability, and a respective reference value; and determining a medical condition of the subject based on the at least one correlation value.

In a 2^{nd} aspect according to the first aspect the pulse wave data indicative of a plurality of heart periods relates to a plurality of heart periods in direct succession to one another.

In a 3^{rd} aspect according to any one of the preceding aspects, the step of determining the respiratory rate variability based on the pulse wave data of the portion of the pulse wave data indicative of a plurality of heart periods comprises: determining a plurality of maxima based on the pulse wave data, the plurality of maxima denoting the maximum amplitude of a respective plurality of heart periods; determining a respiratory signal indicative of the respiratory rate based on the plurality of maxima, optionally including determining the respiratory signal based on a spline interpolation of the plurality of maxima; and determining the respiratory rate variability based on a time difference between each maximum of the respiratory signal.

In a 4^{th} aspect according to any one of the preceding aspects, the step of determining the heart rate variability based on the pulse wave data of the portion of the pulse wave data indicative of a plurality of heart periods comprises: determining a plurality of reference points based on the pulse wave data, the plurality of reference points corresponding to a respective component of the plurality of heart periods, optionally the respective component being one of a maximum amplitude of the heart period, a rising edge of the heart rate amplitude; determining the heart rate variability based on a time difference between each reference point of the plurality of reference points.

In a 5^{th} aspect according to any one of the preceding aspects, the subject has a body height, an age, and a gender, and the step of determining the blood pressure variability comprises determining a plurality of blood pressure values, the step of determining a plurality of blood pressure values comprising, for each respective blood pressure value of the plurality of blood pressure values, each respective blood pressure value being associated with a respective heart period of the plurality of heart periods: —determining a systolic component of the respective heart period; —approximating the systolic component with a first Gaussian function and a second Gaussian function; and —determining a time difference (WWT) between the first and second Gaussian functions; and determining a respective blood pressure value (BP) of the plurality of blood pressure values of the subject based on the time difference (WWT), the body height, and/or the age.

In a 6^{th} aspect according to the preceding aspect,
the step of determining a plurality of blood pressure values comprises, for each blood pressure value of the plurality of blood pressure values: determining a preliminary stiffness index (SIₚ) based on the body height and the time difference (WWT); determining an adjusted stiffness index (SIₐ) based on the preliminary stiffness index (SIₚ) and the age; and determining the blood pressure value (BP) based on the adjusted stiffness index (SIₐ) and a regression model, and/or
the portion of the pulse wave data is indicative of a plurality of heart periods, and wherein the step of determining the time difference (WWT) further comprises: determining the time difference (WWT) for the plurality of successive heart periods as an average value based on the respective time differences determined for the plurality of heart periods; optionally the average value being the median value of the determined respective time differences; and/or
the first and second Gaussian functions have a respective maximum amplitude, the maximum amplitude of the first Gaussian function being greater than or equal to the maximum amplitude of the second Gaussian function; and/or
the first and second Gaussian functions have respective first and second standard deviations (σ₁, σ₂), the first and second standard deviations (σ₁, σ₂) being equal to each other.

In a 7^{th} aspect according to any one of aspects 5 and 6, the step of approximating the systolic component comprises fitting the first and second Gaussian functions to the systolic component using

$$F ( a , b , c , d , f ) = {\sum }_{i = 1}^{N} {( {S}_{i} - ( a \cdot {e}^{- \frac{1}{2} {( \frac{t - b}{c} )}^{2}} + d \cdot {e}^{- \frac{1}{2} {( \frac{t - f}{c} )}^{2}} ) )}^{2} {=}^{!} min$$

with a, b, c, and d being determined using non-linear optimization or curve-fitting.

In an 8^{th} aspect according to any one of aspects 5 to 7, the regression model comprises a regression function ƒ(SIₐ, g)=BP_{sys}, where SIₐ is the adjusted stiffness index (SIₐ), g is the gender of the subject, and BP_{sys} is the blood pressure; and wherein determining the blood pressure value comprises determining the blood pressure value based on the regression function, optionally wherein the regression function comprises a linear function of the type ƒ(x)=ax+b, wherein a ranges from 1 to 20 mmHg/(m/s) and b ranges from 0 to 80 mmHg, more preferably wherein a ranges from 5 to 15 mmHg/(m/s) and b ranges from 20 to 60 mmHg.

In a 9^{th} aspect according to any one of aspects 5 to 8, determining the adjusted stiffness index (SIₐ) is based on an adjustment function ƒ(SIₚ)=SIₐ, where SIₚ is the preliminary stiffness index and SIₐ is the adjusted stiffness index (SIₐ), optionally wherein the adjustment function is a linear function of the type ƒ(x)=cx+d, where c and d are adjustment factors determined based on a plurality of value pairs comprising an age value and an associated stiffness index value; optionally wherein

$$c = \frac{SI - μ}{range ( age )}$$

with μ=0,109*age+3,699 and range(age)=0,1663*age+4,3858−μ, age being the age of the subject, and d=0.

In a 10^{th} aspect according to any one of aspects 5 to 9, determining the systolic component comprises: determining a respective global maximum of the respective heart period; determining the second order derivative of the respective heart period; determining a maximum value of the second order derivative located at least at a predetermined time difference from the global maximum; and defining the systolic component as a portion of the heart period between the start of the heart period and the maximum value; optionally the predetermined time difference to the global maximum being 350 ms or less, further optionally the predetermined time difference to the global maximum being 250 ms or less.

In an 11^{th} aspect according to any one of aspects 5 to 10, determining the preliminary stiffness index (SIₚ) is based on a function

$${SI}_{p} = \frac{h}{WWT} ,$$

where h is the subject height, WWT is the time difference, and SIₚ is the preliminary stiffness index (SIₚ).

In a 12^{th} aspect according to any one of the preceding aspects, the step of determining at least one correlation value is based on the heart rate variability; the step of determining at least one correlation value further comprising: generating, based on a plurality of heart rate variability values, a frequency distribution indicative of the distribution of the plurality of heart rate variability values in the time domain; determining a plurality of expected values; determining an entropy value indicative of a plurality of expected values, the entropy value being indicative of the medical condition of the subject.

In a 13^{th} aspect according to any one of the preceding aspects, the frequency distribution indicative of the distribution of the plurality of heart rate variability values comprises a histogram, optionally wherein the histogram has a bin size of 8 ms.

In a 14^{th} aspect according to any one of the preceding aspects, the portion of the pulse wave data indicative of a plurality of heart periods covers a period of between 2 minutes and 5 minutes; and the step of determining the variabilities of the blood pressure, respiratory rate, and the heart rate variability, is based on substantially all heart beats comprised in the pulse wave data.

In a 15^{th} aspect according to any one of the preceding aspects, the step of determining at least one correlation value is based on the heart rate variability and the respiration rate variability, and wherein the step of determining at least one correlation value comprises detecting a correspondence between the heart rate variability and the respiration rate variability.

In a 16^{th} aspect in accordance with any one of the preceding aspects, the average value is the median value of the determined respective time differences.

In a 17^{th} aspect in accordance with any one of the preceding aspects, the first and second Gaussian functions have a respective maximum amplitude, the maximum amplitude of the first Gaussian function being greater than or equal to the maximum amplitude of the second Gaussian function.

In an 18^{th} aspect in accordance with any one of the preceding aspects, the first and second Gaussian functions have respective first and second standard deviations, the first and second standard deviations being equal to each other.

In a 19^{th} aspect in accordance with any one of the preceding aspects, determining the systolic component comprises determining a respective global maximum of the respective heart period; determining the second order derivative of the respective heart period; determining a maximum value of the second order derivative located at least at a predetermined time difference from the global maximum; and defining the systolic component as a portion of the heart period between the start of the heart period and the maximum value.

In a 20^{th} aspect in accordance with the preceding aspect, the predetermined time difference to the global maximum is 350 ms or less, preferably wherein the predetermined time difference to the global maximum is 250 ms or less.

In a 21^{st} aspect in accordance with any one of the preceding aspects, the apparatus further comprises a light source configured for transmitting light into an extremity of a subject; wherein the means for providing pulse wave data comprises an optical sensor configured for receiving light reflected from blood flow through the extremity.

In a 22^{nd} aspect in accordance with the preceding aspect, the step of receiving the pulse wave data comprises activating the light source and receiving the pulse wave data based on a signal provided by the optical sensor.

In a 23^{rd} aspect in accordance with the preceding aspect, the optical sensor comprises a video sensor, and wherein the step of receiving the pulse wave data further comprises receiving a video stream indicative of the reflected light based on the signal; selecting a region of interest from the video stream containing a plurality of pixels, the region of interest optionally having a size of 50×50 pixels; selecting a plurality of frames from the video stream, each frame of the plurality of frames having a respective time stamp; for each respective frame: —determining, within the region of interest, a first sample value indicative of the sum of the values of a green subcomponent of each pixel of the plurality of pixels; —associating each first sample with the respective time stamp; —generating a first pulse wave from the first samples; and

the step of receiving the pulse wave data further comprising determining a second pulse wave by re-sampling the first pulse wave based on the respective time stamps.

In a 24^{th} aspect in accordance with the preceding aspect, determining the second pulse wave further comprises filtering the second pulse wave using a bandpass filter, the bandpass filter optionally removing all frequencies not falling within a range from 0.6 Hz to 2.5 Hz.

In a 25^{th} aspect in accordance with any one of the preceding aspects, the portion of the pulse wave data is indicative of 1 to 50 heart periods, preferably wherein the portion of the pulse wave data is indicative of 1 to 40 heart periods, more preferably wherein the portion of the pulse wave data is indicative of 10 to 30 heart periods.

In a 26^{th} aspect in accordance with any one of the preceding aspects, the portion of the pulse wave data is indicative of a plurality of successive heart periods.

In a 27^{st} aspect in accordance with any one of aspects 21 to 26, the sensor is an optical sensor and the apparatus further comprises a light source, the sensor being configured to detect a signal emitted by the light source and reflected by part of a body of the subject, optionally the part of the body of the subject comprising a pulsatile blood flow of the subject.

In a 28^{th} aspect in accordance with any one of the preceding aspects, the apparatus further comprises input means configured to receive a user input initiating determining of the blood pressure value.

In a 29^{th} aspect in accordance with any one of the preceding aspects, the apparatus further comprises output means configured to display the blood pressure value.

In a 30^{th} aspect in accordance with any one of the preceding aspects, the means for providing pulse wave data comprises a memory unit configured to store the pulse wave data.

According to the invention, in a 31^{st} aspect there is provided an apparatus for determining a medical condition of a human subject, the apparatus comprising a control unit; and a means for providing pulse wave data representative of a heart beat of the human subject; wherein the control unit is configured to perform the steps of receiving the pulse wave data; selecting a portion of the pulse wave data indicative of a plurality of heart periods; determining a first index indicative of a heart rate variability based on the pulse wave data of the portion of the pulse wave data indicative of a plurality of heart periods; determining a second index indicative of a heart rate variability based on the pulse wave data of the portion of the pulse wave data indicative of a plurality of heart periods, the second index being different from the first index; and determining a medical condition of the subject based on the first and second indexes.

In a 32^{nd} aspect according to the preceding aspect, determining the first index comprises determining a plurality of respiratory rate intervals based on the pulse wave data of the portion of the pulse wave data indicative of a plurality of heart periods; and determining the first index based on the plurality of respiratory rate intervals.

In a 33^{rd} aspect according to the preceding aspect, determining the first index further comprises determining an average based on the plurality of respiratory rate intervals; and determining the first index based on the average.

In a 34^{th} aspect according to the preceding aspect, the average is the root mean square of successive difference, optionally wherein determining the root mean square of successive difference based on the plurality of respiratory rate intervals includes normalizing the root mean square of successive difference based on a mean respiratory rate interval determined based on the plurality of respiratory rate intervals.

In a 35^{th} aspect according to aspect 31, determining the first index comprises the steps of determining a tachogram indicative of a variability of a plurality of respiratory rate intervals based on the pulse wave data of the portion of the pulse wave data indicative of a plurality of heart periods; determining a frequency distribution of respective respiratory rate intervals of the plurality of respiratory rate intervals; determining an entropy value based on the frequency distribution; and determining the first index based on the entropy value.

In a 36^{th} aspect according to the preceding aspect, the frequency distribution comprises a histogram indicative of a plurality of probabilities; optionally wherein the entropy value is determined based on

S=−Σᵢ₌₁pᵢ·log₂(pᵢ),

wherein pi correspond to the plurality of probabilities; further optionally wherein the histogram has a bin size of 8 ms.

In a 37^{th} aspect according to aspect 31, determining the first index comprises the steps of determining a plurality of beat-to-beat intervals (BBI) based on the pulse wave data of the portion of the pulse wave data indicative of a plurality of heart periods; and determining the first index based on the plurality of beat-to-beat intervals.

In a 38^{th} aspect according to the preceding aspect, determining the first index comprises the steps of determining a Poincare Plot Analysis (PPA) based on the plurality of beat-to-beat intervals, the Poincare Plot Analysis being indicative of a time series fluctuation determined based on a respective relationship of a first beat-to-beat interval (BBIn) and a preceding second beat-to-beat interval (BBIn−1) of the plurality of beat-to-beat intervals; and determining the first index based on the Poincare Plot Analysis.

In a 39^{th} aspect according to the preceding aspect, determining the first index comprises the steps of determining a standard deviation SD1 of a short-term beat-to-beat interval variability and a standard deviation SD2 of a long-term beat-to-beat interval variability; and determining the first index based on an index SD1/SD2 indicative of a ratio of the standard deviation SD1 to the standard deviation SD2.

In a 40^{th} aspect according to any one of aspects 31 to 36, determining the second index comprises determining a plurality of beat-to-beat intervals (BBI) based on the pulse wave data of the portion of the pulse wave data indicative of a plurality of heart periods; and determining the second index based on the plurality of beat-to-beat intervals.

In a 41^{th} aspect according to the preceding aspect, determining the second index comprises determining a Poincare Plot Analysis (PPA) based on the plurality of beat-to-beat intervals, the Poincare Plot Analysis being indicative of a time series fluctuation determined based on a respective relationship of a first beat-to-beat interval (BBIn) and a preceding second beat-to-beat interval (BBIn−1) of the plurality of beat-to-beat intervals; and determining the second index based on the Poincare Plot Analysis.

In a 42^{nd} aspect according to the preceding aspect, determining the second index comprises determining a standard deviation SD1 of a short-term beat-to-beat interval variability and a standard deviation SD2 of a long-term beat-to-beat interval variability; and determining the second index based on an index SD1/SD2 indicative of a ratio of the standard deviation SD1 to the standard deviation SD2.

In a 43^{th} aspect according to any one of aspects 31 to 42, the pulse wave data indicative of a plurality of heart periods relates to a plurality of heart periods in direct succession to one another.

In a 44^{th} aspect according to any one of aspects 31 to 43, the portion of the pulse wave data indicative of a plurality of heart periods covers a period of at least 2 minutes; and the steps of determining the first and second indexes is based on substantially all heart beats comprised in the portion of the pulse wave data indicative of a plurality of heart periods.

In a 45^{th} aspect according to the preceding aspect, the portion of the pulse wave data indicative of a plurality of heart periods covers a period of at least 5 minutes.

In a 46^{th} aspect according to any one of aspects 31 to 45, the control unit is further configured to determine, based the pulse wave data of the portion of the pulse wave data indicative of a plurality of heart periods, for each heart period of the plurality of heart periods, whether the respective heart periods is associated with one or more disruptions, and modify the pulse wave data of the portion of the pulse wave data indicative of a plurality of heart periods if the respective heart periods is associated with one or more disruptions so that the respective heart period is no more associated with the one or more disruptions.

In a 47^{th} aspect according to the preceding aspect, the one or more disruptions comprise a premature beat.

Advantages of the apparatus for determining the blood pressure include that the blood pressure can be determined with improved accuracy. Advantages of the apparatus for determining the medical condition of a human subject include that the biological data, for example, the heart rate, the respiratory rate, the blood pressure, and the variabilities thereof, can be determined with improved accuracy.

### BRIEF DESCRIPTION OF DRAWINGS

FIG. 1 illustrates how the stiffness index is determined in accordance with the present invention;

FIG. 2A contains a flow chart of a method for determining blood pressure in accordance with a first embodiment of the invention;

FIG. 2B contains a flowchart for a method for pulse wave analysis in accordance with the present invention;

FIG. 3A illustrates the detection of the respiratory rate in accordance with one embodiment of the invention;

FIG. 3B illustrates the relationship of the heart rate, blood pressure, and respiratory rate, as well as the variabilities thereof, in accordance with one embodiment of the invention;

FIG. 3C illustrates the application of the Shannon Entropy in detecting atrial fibrillation in a subject in accordance with one embodiment of the invention;

FIG. 4 contains a flow chart of a method for recording pulse wave data in accordance with the present invention, using a mobile device;

FIG. 5A illustrates an exemplary mobile device that can be used in accordance with the method of FIG. 4;

FIG. 5B illustrates an interaction of a human subject with the mobile device shown in FIG. 5A;

FIG. 6 illustrates how a series of heart periods is determined based on acquired pulse wave data;

FIG. 7 illustrates how an exemplary adjustment function for adjusting the stiffness index to the age of a subject is determined;

FIG. 8 illustrates how an exemplary regression model for determining the blood pressure of a subject based on the adjusted stiffness index is determined;

FIGS. 9A and 9B illustrate the correlation of the respective blood pressure of a subject (as estimated based on the regression model and the alternative regression model) and the blood pressure of the subject measured using a common blood pressure monitor;

FIG. 10A illustrates the application of RMSSD in detecting AF in a subject in accordance with one embodiment of the invention;

FIG. 10B illustrates the application of the Shannon Entropy in detecting AF in a subject in accordance with one embodiment of the invention;

FIG. 10C illustrates the application of Poincaré Plot Analysis in detecting AF in a subject in accordance with one embodiment of the invention; and

FIGS. 11A and 11B illustrate the application of Poincaré Plot Analysis in detecting AF in a subject in accordance with one embodiment of the invention.

### DETAILED DESCRIPTION

The elasticity of the vascular system influences the pulse wave of a subject. Based on this effect it has become possible to accurately determine (i.e. in the region of 90% accuracy or more) the blood pressure using an advanced form of photoplethysmography based on specific processing of the pulse wave data. Heart rate and respiratory rate can also be determine based on the pulse wave data of a subject.

The detailed analysis of each of these parameters can form the basis for determining individual conditions of a subject. However, it has been found that, given an accurate representation of the pulse wave data and taking measurements at regular intervals or continuously, the analysis of the heart rate (HR) and the heart rate variability, the blood pressure (BP) and the blood pressure variability, and the respiratory rate (RR) and the variability of the respiratory rate can serve to detect a range of medical conditions, such as CAD, AFIB, sleep apnea, depression and others.

The blood pressure and the blood pressure variability can be detected based on an advanced processing of pulse wave data and using the stiffness index. The respiratory rate and the RR variability can be detected based on an advanced processing of pulse wave data. According to the invention, multiple physiological parameters are simultaneously processed using a novel pulse wave analysis and nonlinear methods for signal analysis. No additional peripheral devices are needed except for a smartphone or smart watch. The apparatus is directed at providing an improved accuracy when differentiating between patients in AF and patients in Sinus Rhythm (SR).

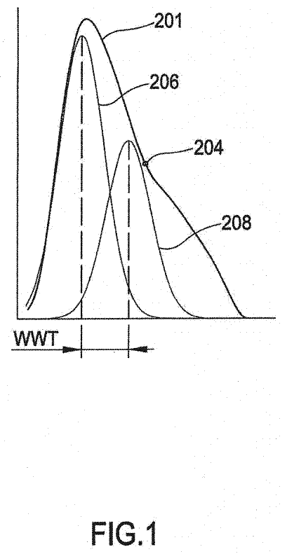

FIG. 1 illustrates how the stiffness index is determined in accordance with the present invention. The diagram in FIG. 1 shows a pulse wave signal 201 over time t as well as corresponding wave components 206 and 208 of the original pulse wave and the wave reflected mainly by the aortic bifurcation. FIG. 1 also shows an inflection point 204. It is noted that a simple partitioning based on the inflection points, as commonly known in the art, does not necessarily correspond to the actual physiological wave components because of the reasons set forth in the previous paragraph. In contrast, in accordance with the present invention, the actual original pulse wave and the wave reflected by the aortic bifurcation are determined by approximation of the graph with Gaussian functions, by which the two component waves w_{original} 206 and w_{reflected} 208 can be obtained with very high accuracy. Here, the time difference is determined as the time difference between the component waves w_{original} and w_{reflected} as opposed to the time difference between two maxima of the graph. This facilitates determining, instead of the commonly known peak-to-peak time (PPT), a wave-to-wave time (WWT), which corresponds to the actual time difference between the original pulse wave and the reflected pulse wave with a substantially higher level of accuracy. This, in turn, facilitates a more accurate calculation of the SI and, thus, leads to an improved correlation with the blood pressure.

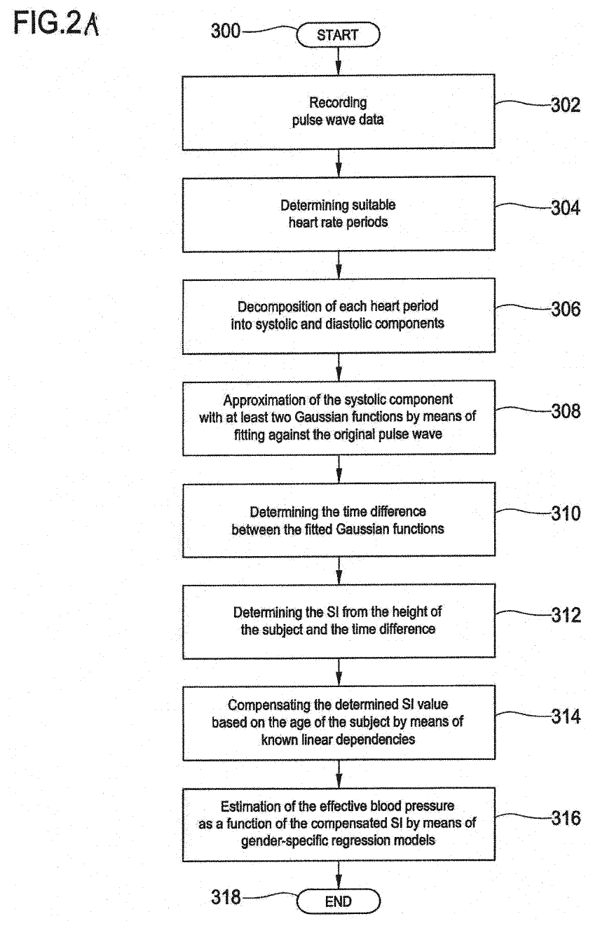

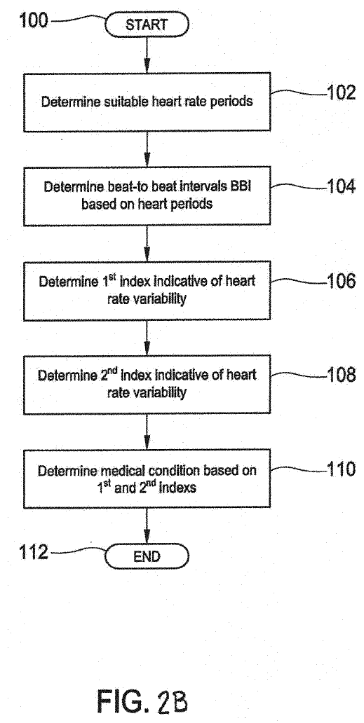

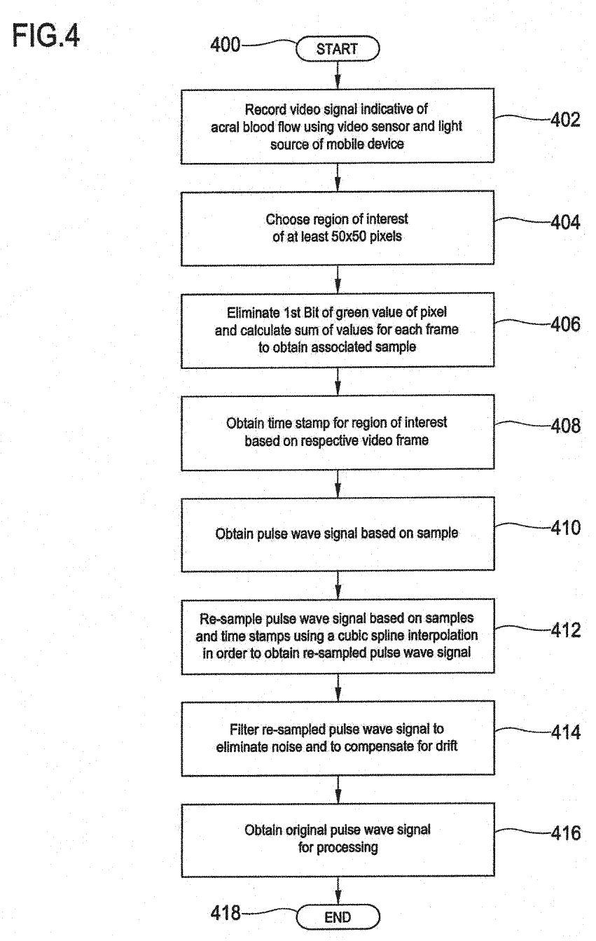

FIG. 2A contains a flow chart of a method 300 for determining blood pressure in accordance with a first embodiment of the invention. In step 302, pulse wave data is recorded. The detection of pulse waves and the recording of data indicative of the detected pulse wave can be performed in any way known in the art. For example, classic photoplethysmography. One example of detection and recording of pulse wave data is described further below with respect to FIG. 4.

In step 304 suitable heart periods are determined. As described above, heart periods vary depending on a number of factors and can exhibit benign (e.g. non-pathological) irregularities, for example caused by stress or anxiety, or consumption of stimulants such as caffeine, nicotine, or alcohol. In order to establish a sound basis for further processing of pulse wave data, suitable heart periods are selected from a longer recording of pule wave data. In the first embodiment, 5 to 30 heart periods are selected from a pulse wave recording of 5 seconds up to 2 minutes in length, provided that all selected heart periods have a similarity to each other of at least 0.8 and are all contained in a single recording segment (i.e. are successive to each other). In other embodiments, a greater or smaller number of successive heart periods may be used, for example 3 to 10 or 20 to 50 heart periods. Further, the recording of pulse wave data can have a different length, for example ranging from 5 to 10 seconds up to 10 to 30 minutes.

In step 306, each heart period is decomposed or partitioned into a systolic and a diastolic component. This is achieved by determining the maximum of the second order derivative of the pulse wave, located at most 350 ms after the systolic maximum. Typically, the maximum of the second order derivative of the pulse wave is located between 250 ms and 350 ms after the systolic maximum. Determining the maximum of the second order derivative is restricted to the above-defined time window in order to take into account the expulsion time of the heart and in order to avoid erroneous detection.

In step 308, an approximation is performed in which the systolic component is approximated by fitting at least two Gaussian functions to the original pulse wave:

$$F ( a , b , c , d , f ) = {\sum }_{i = 1}^{N} {( {S}_{i} - ( a \cdot {e}^{- \frac{1}{2} {( \frac{t - b}{c} )}^{2}} + d \cdot {e}^{- \frac{1}{2} {( \frac{t - f}{c} )}^{2}} ) )}^{2} {=}^{!} min$$

with a, b, c, and d being determined using non-linear optimization. In one embodiment, the two Gaussian functions are fitted to the original pulse wave using the Levenberg-Marquardt algorithm. In this approximation step, the first Gaussian function corresponds to the original pulse wave and the second Gaussian function corresponds to the wave reflected at the aortic bifurcation, whereas the amplitude of the first Gaussian function must be greater or equal to the amplitude of the first Gaussian function, and both functions must exhibit an identical standard deviation σ.

In step 310, the time difference between the two Gaussian functions is calculated as the wave-to-wave time WWT. For example, the WWT can be calculated as the time difference between the base points of the two Gaussian functions. Alternatively, the WWT can be calculated as the time difference between the maxima of the two Gaussian functions. In order to generate an overall or averaged WWTₐ, the median value over 5 to 30 (or any desired number of) heart periods is calculated. This can effectively reduce the impact of outliers.

In step 312, the stiffness index SI is calculated based on the subject height h (in m) and the averaged WWTₐ (in s) as:

$$SI = \frac{h}{{WWT}_{a}}$$

In step 314, the SI value calculated in step 312 is adjusted in order to compensate for the age of the subject. As described above, the elasticity of a person's vascular system decreases with increasing age, so that the average healthy person at an age of 20 necessarily exhibits a lower SI than the average healthy person at an age of 40 or 60. Therefore, the SI is normalized in step 314 in order to achieve comparable results. In the first embodiment, the SI is normalized in order to obtain an age-independent or adjusted SI.

In step 316, the adjusted SI is estimated based on a gender-specific regression model. The gender-specific regression models are the result of proprietary clinical studies and define the estimated blood pressure of a subject as a function of gender and the adjusted SI. In one example, a male person exhibiting an adjusted SI of 10 may have an estimated systolic blood pressure of 180 mm Hg. Clinical studies were conducted in order to determine how the adjusted SI relates to the actual blood pressure depending on the gender of a subject. It has been found that, with male subjects, an adjusted SI of about 10 m/s corresponds to a blood pressure of about 170 mm Hg, and an adjusted SI of about 8 m/s corresponds to a blood pressure of about 150 mm Hg. With female subjects, an adjusted SI of about 10 m/s corresponds to a blood pressure of about 165 mm Hg, and an adjusted SI of about 8 m/s corresponds to a blood pressure of about 155 mm Hg.

In an alternative embodiment, a more comprehensive regression model is applied. In this alternative embodiment, steps 302 to 314 are performed identical to what is described above. In step 316 of the alternative embodiment, however, additional parameters are applied in order to achieve an even higher correlation to the actual blood pressure value. Here, the adjusted SI is estimated based on an alternative regression model that factors in: the gender of the subject (i.e. male or female), an index value I_{f} indicative of the physique of the subject (e.g. the body mass index (BMI) of the person), and an index value Cₜ indicative of a tobacco consumption of the subject (e.g. whether or not the subject is a smoker).

With respect to the index value Cₜ indicative of a tobacco consumption of the subject it is noted that in some embodiments only the current status of a subject is determined, namely whether the subject is currently an active smoker. Studies have shown that a relatively short period of not smoking has an impact on blood pressure in a subject, even if the subject had smoked for an extensive period of time. This effect and related effects can be taken into account by determining the current status of a subject in this manner. In other embodiments the history of the subject can also be taken into account. This can be done by determining a period or periods in which the subject was an active smoker and determining the amount of tobacco consumed in these periods (e.g. number of cigarettes per day). In this manner an individual profile detailing the consumption of tobacco by a subject can be generated and introduced into the regression model. It is noted that long-term tobacco consumption can have multiple effects on the vascular system of a subject, for example regarding stiffness of the blood vessels. Some or all of these effects can be long-term effects that do not disappear during a short period of non-smoking.

One specific alternative regression model, which is also the result of proprietary clinical studies, defines the estimated blood pressure of a subject as a function of the adjusted SI, the gender of the subject (a value of 1 being indicative of a male subject, a value of 2 being indicative of a female subject), the BMI of the subject (the BMI value being calculated based on the height and weight of the subject), and the fact that the subject is a smoker or not (a value of 0 being indicative of the subject not being a smoker, and a value of 1 being indicative of the subject being a smoker). The BMI can be calculated based on

$$BMI = \frac{mass}{{height}^{2}} ,$$

where mass is the weight of the subject in kilograms (kg) and where height is the height of the subject in meters (m). The specific alternative regression model is based on the formula:

BP_{sys}=139.611−19.450·g−0.820 age+0.968·I_{ƒ}+5.394·Cₜ+2.759·SI.

The following table provides further details on the coefficients used in the alternative regression model:

Coefficients (dep. var: BP_{sys})

| | | Non Standardized | | Standardized | | |
|---|---|---|---|---|---|---|
| | | Coefficients | | Coefficients | | |
| | | | Standard | | | |
| Model | | B | Error | Beta | t | Sig. |
| 1 | (Constant) | 139.611 | 40.618 | | 3.437 | 0.004 |
| | Sex | | | | | |
| | (1 = male, | −19,450 | 7.278 | −0.568 | −2.673 | 0.019 |
| | 2 = female) | | | | | |
| | Age | −0.820 | 0.478 | −0.318 | 4.717 | 0.110 |
| | I_{f} (BWII) | 0.968 | 1.513 | 0.140 | 0.639 | 0.534 |
| | Cₜ (Smoker) | 5.394 | 6.830 | 0.144 | 0.790 | 0.444 |
| | SI | 2.759 | 2.286 | 0.228 | 1.207 | 0.249 |

It is noted that the term “physique” of the subject refers to the size, stature, figure, or physique in terms of the absence or presence (and the degree) of adiposity of the subject, i.e. whether the person is overweight or not. Apart from the BMI as described above, there are a number of known methods and/or concepts for quantifying a degree of adiposity in a subject. Examples include, but are not limited to: measuring the percentage of body fat (e.g. by bioelectric impedance analysis, by caliper measurements, or any other known method for determining the percentage of body fat), calculating the waist-to-height ratio, and calculating the waist-to-hip ratio. Bioelectric impedance analysis, for example, can be integrated into household appliances like scales, so that the percentage of body fat can be measured during regular or daily activities, such as stepping on the scale to be weighed. Bioelectric impedance analysis may not be applicable to all subjects due to their individual medical condition, for example when a pace maker or other implant is in place, and/or may not provide the most accurate measurements of body fat percentage. Caliper measurements can be made by a physician or by the subject him/herself by measuring the thickness of a skin fold in order to deduce the body fat percentage. The measurements are typically performed at three or seven different body parts, depending on the method used. Caliper measurements can provide acceptable results but typically cannot accurately measure the percentage of body fat present in and around organs.

It is noted that the alternative regression function described above does not require the use of the BMI in particular, but is, in principle, adaptable to any quantification of a degree of adiposity in a subject. If a measure of a subject's physique other than the subject's BMI is used, a corresponding conversion factor needs to be introduced into the specific formula described above, in order to map the measure to the BMI (or vice versa).

Blood pressure variability is now determined based on a plurality of blood pressure values taken from a subject in the manner described above. Typically, determining blood pressure variability is performed over a period of 2 to 5 minutes, or alternatively, over a number of 120 to 300 heart periods, in order to obtain a representative sample. In other embodiments, however, the determining of blood pressure and blood pressure variability can be performed in a continuous manner, for example using a sliding window of 2 to 5 minutes (or 120 to 300 heart periods).

FIG. 2B contains a flowchart for a method 100 for pulse wave analysis in accordance with the present invention. At step 102, the pulse wave signal is acquired as set forth in more detail below with respect to FIG. 4 and suitable heart periods are selected. Typically, the pulse wave signal is acquired for a time period of at least 2 minutes, preferably at least 5 minutes.

At step 104, a combination of morphology and frequency analysis of the pulse wave is applied to detect all Beat-to-Beat-Intervals (BBI). The applied algorithm yields an improved correlation of r>0.99, compared to RR intervals from standard ECG recordings, which were done in comparison. From the extracted BBI time series, several indices representing the variability of heart rhythm can be calculated and analyzed regarding their ability to discriminate between AF and SR. For the analysis, premature beats and other disruptions can be eliminated and corresponding points on the BBI time series can be replaced, using an algorithm for adaptive variance estimation. The impact of ectopy on variability indices is rather low. However, even in groups exhibiting a minor number of ectopic beats (e.g., less than 5%), filtering of the tachogram can further reduce the impact of ectopy.

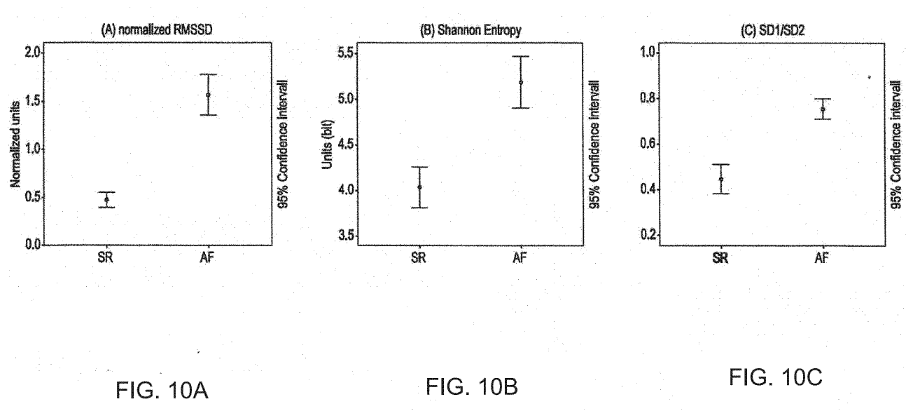

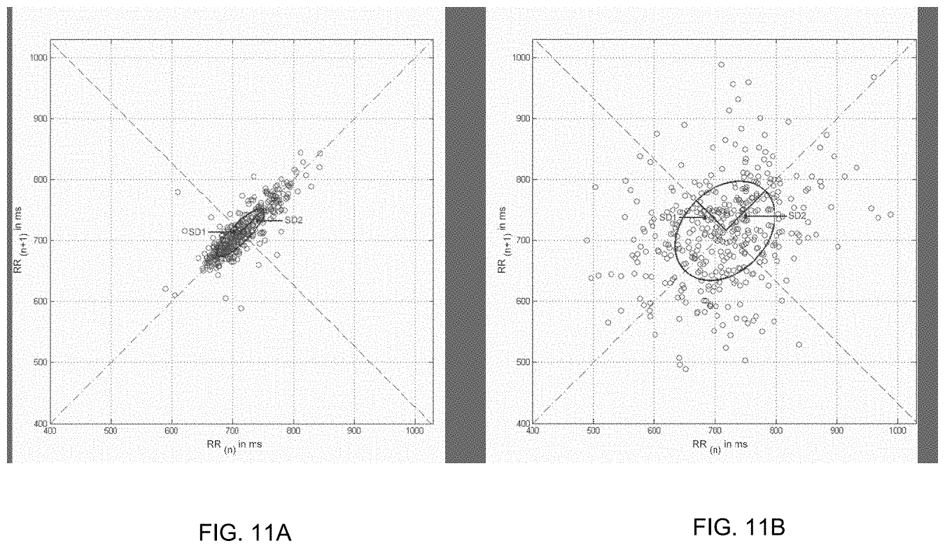

At steps 106 and 108 first and second indexes are determined in accordance with what is described with respect to FIGS. 10A, 10B, and 10C below (see also description of FIGS. 11A and 11B below). In one embodiment, the first index is a root mean square of successive difference (RMSSD) of RR intervals and the second index is an SD1/SD2 index. In other embodiments, other combinations, for example including an index determined based on the Shannon Entropy, can be employed.

At step 110 the medical condition of the subject is determined, based on the first and second indexes. The method 100 ends at step 112.

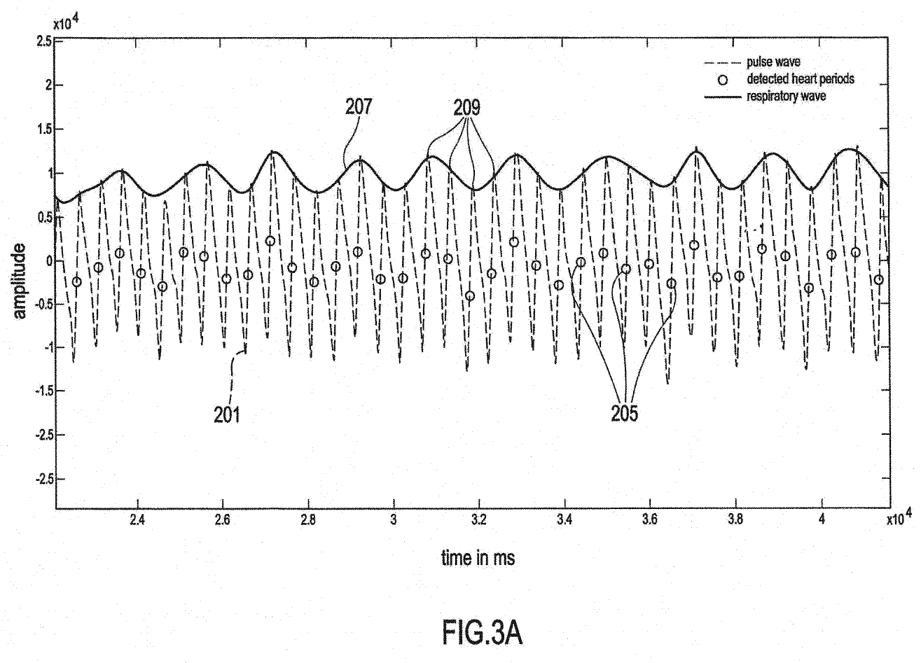

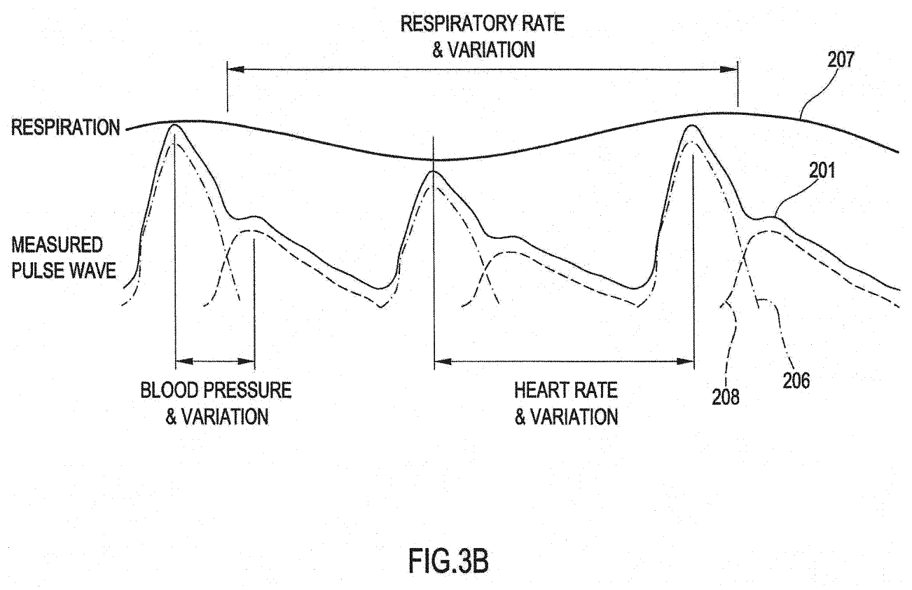

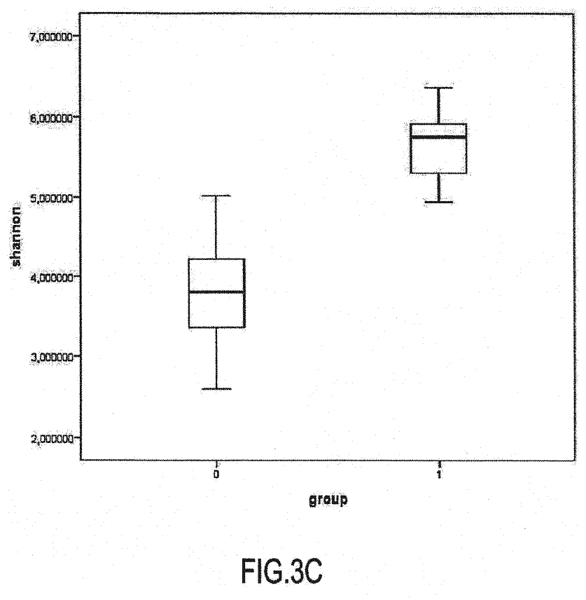

FIG. 3A illustrates the detection of the respiratory rate in accordance with one embodiment of the invention. FIG. 3A shows, on the vertical axis, the amplitude a detected pulse wave 201 over time (see horizontal axis). The pulse wave 201 exhibits an amplitude of between approximately −1 and 1, and the instances of rising edges are registered as detected heart periods 205. Further, a signal 207 indicative of the respiration of the subject is detected based on the maxima 209 of the pulse wave 201.

In order to obtain the signal, the maxima 209 of the pulse wave are sampled using a cubic spline interpolation similar to the re-sampling of the pulse-wave described with respect to FIG. 4 below. Here, two subsequent samples are interpolated by a third-degree polynomial. The position (in time) of the samples corresponds to the time stamps. The polynomial Rᵢ for the range [tᵢ, tᵢ₊₁] is calculated as follows:

Rᵢ=aᵢ+bᵢ(t−tᵢ)²+dᵢ(t−tᵢ)³

with i=1, . . . , n−1. The process of re-sampling includes incrementing t continuously by 1 ms, corresponding to a sample rate of 1000 Hz. In an alternative embodiment, the re-sampling includes incrementing t continuously by 10 ms, corresponding to a sample rate of 100 Hz. The parameters aᵢ, bᵢ, cᵢ, and dᵢ have to be set to suitable values. The pulse wave is obtained as the respiration R, i.e. signal 207, being the result of the sampling. The variation of the respiration rate is then determined based on signal 207 by known methods, for example by detecting a series of maxima of signal 207 and determining a time difference for each pair of subsequent maxima.

FIG. 3B illustrates the relationship of the heart rate, blood pressure, and respiratory rate, as well as the variabilities thereof, in accordance with one embodiment of the invention. FIG. 3B shows a combination of a number of signals determined based on the pulse wave 201. Here, the respiratory rate and the variation thereof are shown based on signal 207. Further, the blood pressure and variation thereof are shown based on pulse wave 201 and the components 206 and 208 thereof, as described above and as shown in FIG. 1. The heart rate and variation thereof is also shown based on pulse wave 201.

Based on an analysis of the heart rate (HR) and the heart rate variability, the blood pressure (BP) and the blood pressure variability, and the respiratory rate (RR) and/or the variability of the respiratory rate, all obtained based on the pulse wave 201 and exhibiting an accuracy previously not obtainable, a range of medical conditions, such as CAD, AF, sleep apnea, depression and others.

Based on the data obtained, AFIB can be detected by analyzing the interaction between heart rate and blood pressure using nonlinear interaction dynamics, for example joint symbolic dynamics (JSD) and segmented Poincaré plot analysis (SPPA). SPPA can be applied to analyze the interaction between two time series—here heart rate and blood pressure. Introducing a parameter set of two indices, one derived from JSD and one from SPPA, the linear discriminant function analysis revealed an overall accuracy of 89% (sensitivity 91.7%, specificity 86.7%) for the classification between patients with stable sinus rhythm (group SR, n=15) and with AF recurrence (group REZ, n=12). The coupling of heart rate and systolic blood pressure provides a potential tool for the prediction of AF recurrence after CV and could aid in the adjustment of therapeutic options for patients with AF.

In a similar manner, depression can be detected by analyzing the relationship between respiration and heart rate and by detecting that respiration and heart rate are not in sync and/or the heart rate does not change upon substantial variation of the respiratory rate. Likewise, sleep apnea can be detected using the above-described mechanisms by analyzing the respiratory rate, typically showing an unusually high variation, and by analyzing the heart rate, typically slowing down during periods of sleep apnea.

FIG. 3C illustrates the application of the Shannon Entropy in detecting atrial fibrillation in a subject in accordance with one embodiment of the invention. Based on the pulse wave, a tachogram is determined, which is indicative of the variations in respiratory rate over time. From the tachogram, a histogram is generated, which represents the frequency distribution of the respiratory rate variations. In one embodiment, the histogram has a bin size of 8 ms, which means that the frequency distribution is based on a discrete time scale divided into 8 ms slots. Each respiratory variation (i.e. between two maxima of signal 207) is sorted into the respective bin. The probabilities represented by the histogram are then used as input for calculating the Shannon Entropy as

S=—Σᵢ₌₁pᵢ·log₂(pᵢ).

The result is a bit value, which determines whether or not a subject belongs to a group of healthy patients or not, whereas a threshold value of 4.8 bits is used:

$$AFib = \{ \begin{matrix}S \geq 4 , 8 bit , then yes \\ otherwise no\end{matrix}$$

It is noted that the above is one example to determining an entropy value for the respiratory rate variations. Other known methods can be used in a similar manner, by simply adapting the threshold value according to the method and calculation used. FIG. 3C illustrates the threshold value of 4.8, clearly distinguishing between subjects showing AFIB (right; value “1”) and not showing AFIB (left; value “0”). One advantage of determining a frequency distribution in the manner described is that the entropy measure is independent from a resting heart rate of the subject. The above is, thus, equally applicable to subjects of all age groups, for example children as well as the elderly, despite of substantial differences in their respective resting heart rates.

FIG. 4 contains a flow chart of a method 400 for recording pulse wave data in accordance with the present invention, using a mobile device having video recording capabilities. Mobile communication devices, in particular so-called smart phones, have extensive capabilities beyond mere telecommunication. For example, most mobile phones are typically provided with a digital camera capable of capturing still images and video and with a corresponding light source for low-light situations. In general, to record a pulse wave by detecting, with an optical sensor, light emitted from a light source and reflected by a finger of a subject. In one embodiment, pulse wave data is obtained using a common mobile device equipped with a digital camera (e.g. used as an optical sensor) and an LED flashlight (e.g. used as a light source). The light emitted by the light source is reflected and the properties of the light (e.g. intensity, hue, brightness, saturation) are affected (e.g. one or more of the properties are modulated) by the acral blood flow.

In step 402, the subject places their finger on both the light source and the camera of the mobile device such that light emitted from the light source illuminates the acral blood flow and is reflected and detected by the camera. The video signal thus created is recorded and stored in a memory unit of the device. Alternatively, the video signal (e.g. a video stream) can be processed directly, without necessitating storing the pulse wave data in a memory unit.

In step 404, a region of interest (ROI) is selected from the full resolution video stream. This selection can be performed, for example, based on brightness information contained in the video stream. In one embodiment, the ROI is determined in a region of maximum brightness within a video frame, off the center and at a minimum distance from the border. This can ensure that a region is chosen that is sufficiently illuminated (e.g. a region close to the light source). In one embodiment, the ROI has a size of at least 50×50 pixels (i.e. 2500 square pixels). Generally, the ROI can have a size ranging from 625 to 10000 square pixels, preferably 900 to 6400 square pixels, more preferably 1600 to 3200 square pixels.

In step 406, for the ROI of each frame of the video stream, a sample sᵢ is calculated, based on

$${s}_{i} = {\sum }_{j = 0}^{N - 1} {\sum }_{k = 0}^{M - 1} \frac{p ( j \cdot w + k )}{2}$$

with p being the value of the green channel of the pixel located within the ROI at the position j, k; N and M being the size of the ROI; and w being the width of the ROI. The division by 2 eliminates the lowest Bit of p, such that noise is effectively reduced. This produces a sample s₁ for each captured video frame. In alternative embodiments, a different channel or channels (e.g. red, blue, or a combination of red, green, and/or blue) can be employed instead of the green channel. This may also depend upon the individual device used (e.g. make and model of smartphone, smart watch).

In step 408, a time stamp tᵢ is generated for each sample sᵢ (more accurately, for each video frame, based on which the sample was calculated) and encoded into the video stream by the video camera.

In step 410, the pulse wave is obtained as a pulse wave signal based on the samples sᵢ obtained in step 406.

In step 412, a re-sampled pulse wave is obtained by re-sampling the pulse wave from the samples sᵢ (i.e. as obtained in step 410) based on the associated time stamps obtained in step 408. This is necessary due to technical issues in detecting, generating, and encoding video data, for example resulting in dropped frames or non-constant frame rates. Based on these issues, the samples sᵢ cannot be obtained at fixed and reliable time intervals. In order to obtain the re-sampled pulse wave, the pulse wave is re-sampled using a cubic spline interpolation and is performed on each polynomial. Here, two subsequent samples are interpolated by a third-degree polynomial. The position (in time) of the samples corresponds to the time stamps. The polynomial Sᵢ for the range [tᵢ, tᵢ₊₁] is calculated as follows:

Sᵢ=aᵢ+bᵢ(t−tᵢ)²+dᵢ(t−tᵢ)³

with i=1, . . . , n−1. The process of re-sampling includes incrementing t continuously by 1 ms, corresponding to a sample rate of 1000 Hz. The parameters aᵢ, bᵢ, cᵢ, and dᵢ have to be set to suitable values. The pulse wave is obtained as the signal S being the result of the re-sampling. In an alternative embodiment, the re-sampling includes incrementing t continuously by 10 ms, corresponding to a sample rate of 100 Hz.

In step 414, the re-sampled pulse wave is filtered to eliminate noise and to compensate for drift. This can be achieved by applying a common bandpass filter (e.g. 0.1 to 10 Hz).

In step 416, the original pulse wave signal is obtained in order to be processed further, as described above with respect to FIG. 3 (see, e.g., steps 304 ff.)

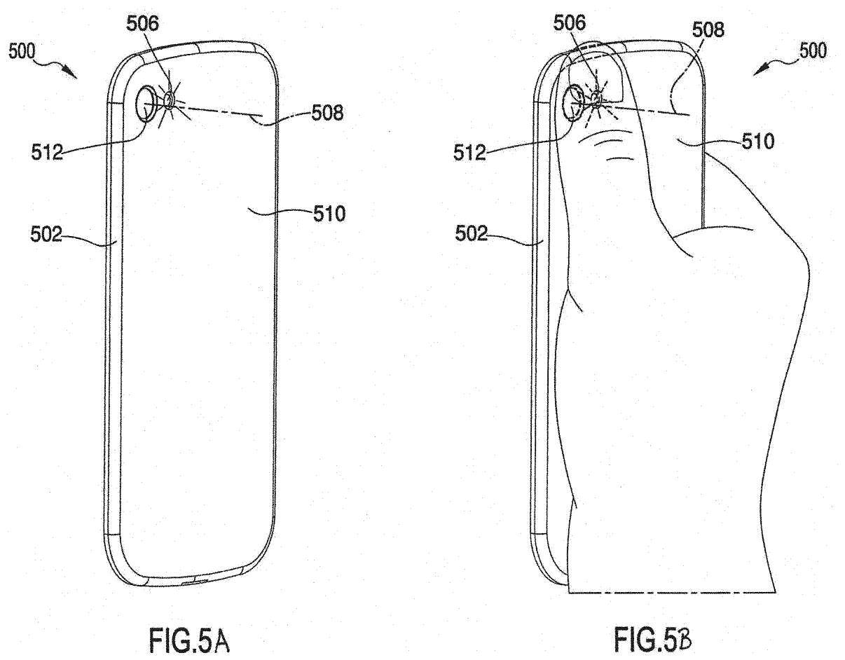

FIG. 5A illustrates a exemplary mobile device that can be used in accordance with the method of FIG. 4. The mobile device 500 has a frame or main body 502 and a device panel 510. In some examples, the device panel 510 can be a back panel of the mobile device 500. The device 500 further has a camera device 512 capable of detecting digital video signals, for example in the form of digital still images and digital video. The camera device 512 is configured to detect video signals representative of objects located generally with a frustum-shaped region along a main detection direction 508. The device 500 further has a light source 506 configured to illuminate any objects located in front of camera device 512, i.e. located within the frustum-shaped region and/or along a main detection direction 508. The light source 506 can be configured to provide both a single flash of light and a continuous light beam, depending on a mode of operation. When recording video, the light source typically provides a continuous light beam. Light emitted from light source 506 will be reflected by an object placed within the view of camera device 512 so that the reflected light can be detected by camera device 512. Mobile device 500 further comprises a control unit (e.g. a CPU, micro processor, SoC; not shown) coupled to other components, such as camera device 512, light source 506, a memory unit, a user interface, input means, an audio unit, a video unit, a display, and other.

FIG. 5B illustrates an interaction of a human subject with the mobile device shown in FIG. 5A. In order to take a measurement, the subject places a finger (e.g. a thumb) on mobile device 500, covering both the camera device 512 and the light source 506. The individual configuration of the mobile device (e.g. a position of camera device 512 and light source 506 or the distance in between) is of secondary relevance, as long as it is physically possible to cover both the camera device 512 and the light source 506 with a suitable extremity (e.g. finger, thumb, ear). In this respect, any extremity suitable for (acral) measurement can be used in accordance with the present invention. In general, any body part that is associated with pulsating blood flow can be used in accordance with the present invention, as long as a meaningful signal indicative of the blood flow can be detected via the body part. In some embodiments, the control unit of mobile device 500 will process signals provided by camera device 512 and detect, based on the signals provided, that one or more parameters indicative of video quality (e.g. brightness, contrast, focus) are outside of preferred operating ranges due to the low-light and/or close-proximity situation created by the placement of the thumb directly onto camera device 512. The control unit may then provide control signals to one or more components, for example to light source 506, in order to make adjustments to the parameters (e.g. activating light source 506 in order to compensate for low light).

Upon placement of the suitable extremity (here, e.g., the thumb of the subject), the measurement is initiated by activating the light source 506 to emit a continuous light beam of sufficient intensity, such that acral blood flow is illuminated. At substantially the same time, camera device 512 is activated and the light reflected by the acral blood flow is detected by camera device 512. Both activating the light source 506 and activating the camera device 512 can be achieved by corresponding program code executed by the control unit comprised in device 500. The activation can be triggered manually, for example by selecting a corresponding function on a user interface of device 500, or automatically, for example triggered by a sensor (e.g. a proximity sensor, an optical sensor), a timer, voice recognition, or other (input means). In one example, the signal of the sensor is continuously processed to check for the presence of a suitable signal. Video data is then recorded or transmitted for further processing for a predetermined period of time, typically ranging from several seconds to 2 minutes. In some embodiments, the time period is not predetermined, but determined as the recording/transmitting is ongoing, in that a quality measure is calculated from the recorded/transmitted data and the recording/transmitting is performed until a sufficient number of heart periods (e.g. 10-30) of sufficient quality (e.g. similarity; see in further detail below) has been recorded/transmitted. Completion of the recording/transmitting can be indicated to the subject, for example, by an acoustic and/or optical signal emitted by an audio and/or video component of device 500.

It is noted that other embodiments employ the same or different sensors and/or devices. For example, smart watches having a corresponding light source/sensor assembly as described above with respect to FIGS. 5A and 5B, can be used as well. These devices have an advantage in that the sensor is kept in close proximity to the body (here: wrist) of a subject, thereby facilitating continuous measurements and/or measurements of arbitrary duration and at arbitrary time points, without interaction of a subject (e.g. also during sleep). It is noted that the above concepts apply to a range of sensors and are not limited to a particular or otherwise specific embodiment of sensor hardware.

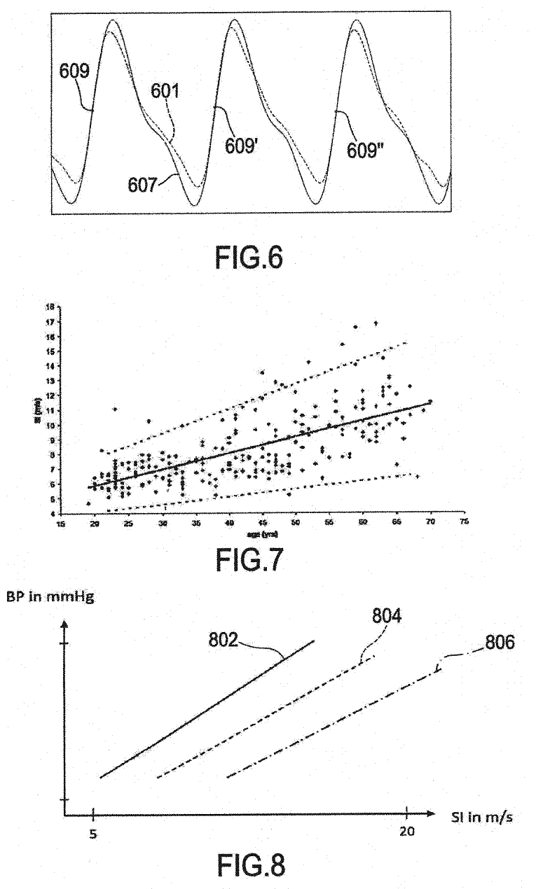

FIG. 6 illustrates how a series of heart periods is determined based on acquired pulse wave data 601. Pulse wave data can be acquired from live measurements taken with a human subject or can be retrieved from data storage when measurements recorded at an earlier time are to be processed. Pulse wave data 601 contains signals corresponding to a number of heart periods exhibited by the subject over an extended time period. In some examples, the pulse wave data cover several minutes of recorded heart periods, for example 2 minutes, preferably several seconds to 2 minutes. In other examples, the pulse wave data can cover substantially less (e.g. 5-30 seconds) or more (several hours) of recorded heart periods. For reasons of clarity, FIG. 6 shows merely three successive heart periods representing only a small window of pulse wave data covering an extended period of time of typically up to 2 minutes.

The pulse wave data 601 is partitioned into single heart periods by generating an amplified wanted signal 607 from the original pulse wave 601 and scanning the amplified signal for rising edges. In general, a pulse wave comprising a single heart period is sufficient, but typically a pulse wave comprising a plurality of successive heart periods is used. In detail, a spectrum is created from the filtered (see FIG. 4, step 414, and corresponding description above) pulse wave signal 601 using discrete Fourier transformation (DFT): Spec=|DFT(S_{filter})|. In this spectrum, the maximum frequency in the range of 0.6 Hz to 2.5 Hz is determined and regarded as the dominant heart frequency: idx=argmax{Spec_{range}}, wherein Spec_{range} corresponds to the spectrum from 0.6 Hz to 2.5 Hz and idx corresponds to the index (i.e. frequency) in the spectrum. Then, a normalized Gaussian graph having values in the range [0,1] is superposed over the dominant heart frequency and over the 2 harmonic components thereof, such that a minor variation of the heart rate is accounted for. The standard deviation a of the Gaussian graphs should intersect at 3σ, with:

$$σ = \frac{idx}{6} and gauss ( t ) = {e}^{- \frac{1}{2} {( \frac{t - idx}{σ} )}^{2}} + {e}^{- \frac{1}{2} {( \frac{t - 2 \cdot idx}{σ} )}^{2}} + {e}^{- \frac{1}{2} {( \frac{t - 3 \cdot idx}{σ} )}^{2}} .$$

The wanted signal is obtained by multiplying the spectrum with the Gaussian function and subsequent back transformation: S_{wanted}=Real(IDFT (Spec·gauss)). The amplified signal Sₐₘₚ is then obtained by multiplication of the wanted signal and addition to the original signal:

$${S}_{amp} = \frac{1}{2} ( \frac{{S}_{filter}}{f} + f \cdot {S}_{nutz} ) ,$$

with ƒ being the amplification factor. Subsequently, the first order derivative of the amplified signal Sₐₘₚ is calculated and the maxima thereof, indicating the inflection points on the amplified signal Sₐₘₚ, and, thus, the rising edges thereof. This provides the location of each heart period, defined between the two local minima before and after the rising edge of each heart period.

For a successive number of heart periods, a similarity score is then determined. A cross correlation of each heart period with a template heart period Pₜₑₘₚₗₐₜₑ is calculated and a predetermined number of heart periods (e.g. 10 heart periods) having the highest correlation is obtained. In one embodiment, the similarity (i.e. correlation) of successive heart periods is 0.9 or greater. If each heart period of a minimum number of successive heart periods (e.g. 10-30) fulfills the similarity requirement, then a portion of the pulse wave suitable for further processing has been identified.

FIG. 7 illustrates how an exemplary adjustment function for adjusting the stiffness index to the age of a subject is determined. The horizontal axis of the graph indicates the age of a subject (in years) and the vertical axis indicates the SI (in m/s). The distribution of measured SI of a number of subjects and a correlation with the age of the respective subject provides a statistical basis for computing the adjustment function as shown in FIG. 7. Here, the SI of a subject being 60 or 65 years of age can be correlated to the SI of a subject being 20 or 25 years of age.

FIG. 8 illustrates how an exemplary regression model for determining the blood pressure of a subject based on the adjusted stiffness index is determined. The regression model is age-dependent in that regression line 802 serves to provide a regression function for subjects aged 20 to 30 years. In the same manner, regression lines 804 and 806 serve to provide regression functions respectively for subjects aged 30 to 40 years and 60 to 70 years. The regression model facilitates associating the SI of a subject belonging to a particular age group to a corresponding blood pressure value. As the data underlying the regression model is updated, the regression model can be adjusted over time in order to improve the accuracy thereof.

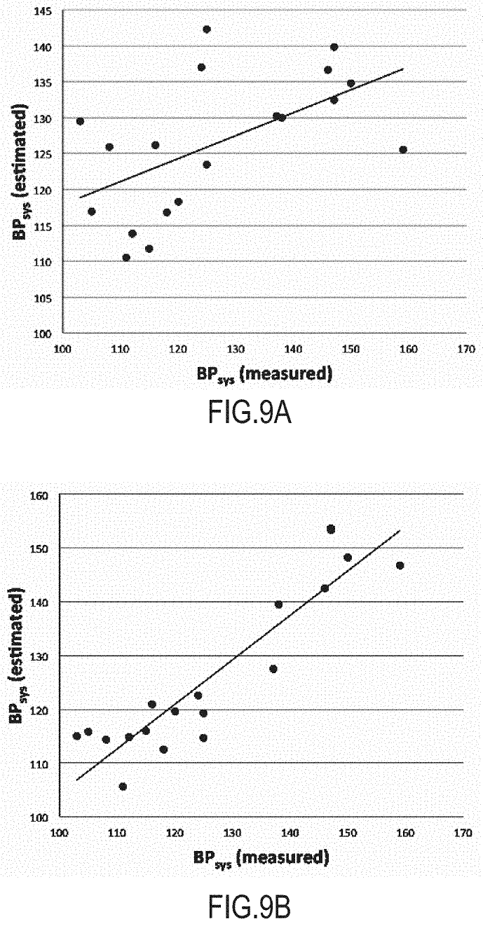

FIGS. 9A and 9B illustrate the correlation of the respective blood pressure of a subject (as estimated based on the regression model and the alternative regression model) and the blood pressure of the subject measured using a common blood pressure monitor. FIG. 9A illustrates the correlation of the estimated blood pressure of a subject and the blood pressure of the subject measured using a common blood pressure monitor. The blood pressure was estimated based on the regression model described above and the correlation was R=0.57. FIG. 9B illustrates the correlation of the estimated blood pressure of a subject and the blood pressure of the subject measured using a common blood pressure monitor. The blood pressure was estimated based on the alternative regression model described above and the correlation was R=0.91.

FIG. 10A illustrates the application of RMSSD in detecting AF in a subject in accordance with one embodiment of the invention. The RMSSD is a standard index from heart rate variability (HRV) analysis to quantify beat-to-beat alterations. In order to adjust for the effect of heart rate on the RR variability the RMSSD value is normalized to the mean RR interval value. Since in AF the variability is distinctly higher than in SR, normalized RMSSD is expected to be higher in patients with AF. In a first comparative embodiment, normalized RMSSD and Shannon Entropy (ShE) were combined. Both indices were extracted from the pulse wave tachogram. Sensitivity, specificity and accuracy were calculated for each of these indices separately and for the combination. For the discrimination between AF and SR based on a two minute pulse wave recording the ShE yielded a sensitivity and specificity of 85% and 95% respectively, applying a cut-off value of 4.9 (see FIG. 10B). This translates into 34/40 patients classified correctly and 2/40 patients classified incorrectly as AF.

FIG. 10B illustrates the application of the Shannon Entropy in detecting AF in a subject in accordance with one embodiment of the invention. Shannon Entropy (ShE) is a statistical method to quantify uncertainty for a random variable and is expected to be higher in patients with AF since the pulse in these circumstances exhibits greater RR interval irregularity compared to pulses recorded from patients with SR.

Based on the pulse wave, a tachogram is determined, which is indicative of the variations in respiratory rate over time. From the tachogram, a histogram is generated, which represents the frequency distribution of the respiratory rate variations. In one embodiment, the histogram has a bin size of 8 ms, which means that the frequency distribution is based on a discrete time scale divided into 8 ms slots. Each respiratory variation (i.e. between two maxima of signal 207) is sorted into the respective bin. The probabilities represented by the histogram are then used as input for calculating the Shannon Entropy as

S=−Σᵢ₌₁pᵢ·log₂(pᵢ).

The result is a bit value, which determines whether or not a subject belongs to a group of healthy patients or not, whereas a threshold value of 4.9 bits is used:

$$AFib = \{ \begin{matrix}S \geq 4.9 bit , then yes \\ otherwise no\end{matrix}$$

It is noted that the above is one example to determining an entropy value for the respiratory rate variations. Other known methods can be used in a similar manner, by simply adapting the threshold value according to the method and calculation used. FIG. 10C illustrates the threshold value of 4.9, clearly distinguishing between subjects showing AF (right) and subjects showing SR (left). One advantage of determining a frequency distribution in the manner described is that the entropy measure is independent from a resting heart rate of the subject. The above is, thus, equally applicable to subjects of all age groups, for example children as well as the elderly, despite of substantial differences in their respective resting heart rates.

In a second comparative embodiment, a filter was applied to the pulse wave tachogram to eliminate premature beats and other disruptions as described above. This improved the applicability of the method and allowed patients with premature beats to be successfully separated from patients with AF. The application of the tachogram filter improved sensitivity to 87.5% while specificity remained stable at 95% using the index normalized RMSSD with a cut-off at 0.09. This translates into 35/40 patients classified correctly and 2/40 patients classified incorrectly as AF.

FIG. 10C illustrates the application of Poincaré Plot Analysis (PPA) in detecting AF in a subject in accordance with one embodiment of the invention. In a third comparative embodiment, an additional index SD1/SD2 that was extracted from a Poincaré Plot of a five minute recording was tested. SD1/SD2, normalized RMSSD and Shannon Entropy were calculated from a filtered tachogram. Sensitivity, specificity and accuracy were then calculated for each method separately and for the combination. By prolonging the recording time from two to five minutes, and combining the index SD1/SD2 and normalized RMSSD, sensitivity and specificity increased to 95% with an area under the curve of 0.93 (see FIGS. 11A and 11B). The cut-off for classification as AF was a normalized RMSSD>0.043 and a SD1/SD2>0.6. This translates into 38/40 patients classified correctly and 2/40 patients classified incorrectly as AF.

It was found that the highest sensitivity and specificity was achieved using the combination of the indices normalized RMSSD and SD1/SD2 with the tachogram filter (see third comparative embodiment) in combination with prolonging the analyzed interval from two to five minutes. Consequently, a sensitivity and specificity of 95% was achieved.

The results are based on a group of eighty patients included in a study (AF 40 pts, SR at time of examination 40 pts). Patients in the AF group had a mean age of 80 years (SD±8), patients in the SR group 75 years (SD±7). Male to female ratio was 2.4 in the AF group and 2.5 in the SR group. The average RR-interval was higher in the AF group. (AF 887±120 ms and SR 784±144 ms, p=0.0004). The results of the comparative embodiments are shown in the following table:

TABLE 1

| | SR | AF | | | | |
|---|---|---|---|---|---|---|
| | (mean ± SD) | (mean ± SD) | p value | AUC | Sensitivity | Specificity |
| Method 1 | | | | | | |
| nRMSSD | 0.103 ± 0.093 | 0.298 ± 0.121 | <0.001 | 0.892 | 50% | 95% |
| ShE | 3.858 ± 0.711 | 5.350 ± 0.825 | <0.001 | 0.912 | 85% | 95% |
| nRMSSD + ShE | — | — | — | 0.917 | 82.5% | 95% |
| Method 2 | | | | | | |
| nRMSSD | 0.034 ± 0.026 | 0.146 ± 0.067 | <0.001 | 0.938 | 87.5% | 95% |
| ShE | 3.710 ± 0.643 | 5.007 ± 0.790 | <0.001 | 0.911 | 77.5% | 95% |
| nRMSSD + ShE | — | — | — | 0.926 | 87.5% | 95% |
| Method 3 | | | | | | |
| nRMSSD | 0.039 ± 0.026 | 0.154 ± 0.070 | <0.001 | 0.942 | 77.5% | 95% |
| ShE | 4.030 ± 0.697 | 5.187 ± 0.885 | <0.001 | 0.872 | 57.5% | 95% |
| SD1/SD2 | 0.447 ± 0.202 | 0.757 ± 0.141 | <0.001 | 0.903 | 77.5% | 90% |
| nRMSSD + ShE | — | — | — | 0.966 | 80% | 95% |
| ShE + SD1/SD2 | — | — | — | 0.959 | 50% | 95% |
| nRMSSD + SD1/SD2 | — | — | — | 0.931 | 95% | 95% |

FIGS. 11A and 11B illustrate the application of Poincaré Plot Analysis (PPA) in detecting AF in a subject in accordance with one embodiment of the invention. PPA provides a visual tool to characterize the complex nature of time series fluctuations where BBIₙ is plotted against BBIₙ₋₁. The Poincaré plot usually displays an elongated cloud of points oriented along the diagonal of the coordinate system. An ellipse is fitted to the cloud of points to characterize its shape. The index SD1/SD2 represents the ratio of the standard deviation of short-term BBI variability (axis vertical to the line of identity, SD1) to the standard deviation of the long-term BBI variability (axis along the line of identity, SD2). The index shown was extracted from five minutes recordings to ensure the formation of the ellipse.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and the scope of the appended claims.

1.-30. (canceled)

31. An apparatus for determining a medical condition of a human subject, the apparatus comprising:
a control unit; and
a sensor unit configured to provide pulse wave data representative of a heart beat of the human subject; wherein the control unit is configured to perform operations comprising:
receiving the pulse wave data;
selecting a portion of the pulse wave data indicative of a plurality of heart periods;
for the selected portion of the pulse wave data indicative of a plurality of heart periods:
determining a blood pressure variability based on the pulse wave data of the portion of the pulse wave data indicative of the plurality of heart periods,
determining a respiratory rate variability based on the pulse wave data of the portion of the pulse wave data indicative of the plurality of heart periods, and
determining a heart rate variability based on the pulse wave data of the portion of the pulse wave data indicative of the plurality of heart periods; and
determining at least one correlation value based on at least one of the blood pressure variability, the respiratory rate variability, the heart rate variability, and a respective reference value; and
determining a medical condition of the human subject based on the at least one correlation value.

32. The apparatus according to claim 31, wherein the pulse wave data indicative of the plurality of heart periods relates to a plurality of heart periods in direct succession to one another.

33. The apparatus according to claim 31, wherein determining the respiratory rate variability based on the pulse wave data of the portion of the pulse wave data indicative of the plurality of heart periods comprises:
determining a plurality of maxima based on the pulse wave data, the plurality of maxima denoting the maximum amplitude of a respective plurality of heart periods;
determining a respiratory signal indicative of the respiratory rate based on the plurality of maxima; and
determining the respiratory rate variability based on a time difference between each maximum of the respiratory signal.

34. The apparatus according to claim 33, wherein determining the respiratory signal includes including determining the respiratory signal based on a spline interpolation of the plurality of maxima.

35. The apparatus according to claim 31, wherein determining the heart rate variability based on the pulse wave data of the portion of the pulse wave data indicative of a plurality of heart periods comprises:
determining a plurality of reference points based on the pulse wave data, the plurality of reference points corresponding to a respective component of the plurality of heart periods;
determining the heart rate variability based on a time difference between each reference point of the plurality of reference points.

36. The apparatus according to claim 35, wherein the respective component is at least one of a maximum amplitude of the heart period and a rising edge of a heart rate amplitude.

37. The apparatus according to claim 31, wherein the human subject has a body height, an age, and a gender, and
wherein determining the blood pressure variability comprises determining a plurality of blood pressure values, wherein each respective blood pressure value is associated with a respective heart period of the plurality of heart periods, and determining each of the plurality of blood pressure values comprises:
determining a systolic component of the respective heart period,
approximating the systolic component with a first Gaussian function and a second Gaussian function; and
determining a time difference (WWT) between the first and second Gaussian functions, and
determining a respective blood pressure value (BP) of the plurality of blood pressure values of the human subject based on the time difference (WWT), the body height, and the age.

38. The apparatus according to claim 37, wherein determining the plurality of blood pressure values comprises, for each blood pressure value of the plurality of blood pressure values:
determining a preliminary stiffness index (SIₚ) based on the body height and the time difference (WWT);
determining an adjusted stiffness index (SIₐ) based on the preliminary stiffness index (SIₚ) and the age; and
determining the blood pressure value (BP) based on the adjusted stiffness index (SIₐ) and a regression model,

39. The apparatus according to claim 38, wherein the portion of the pulse wave data is indicative of a plurality of successive heart periods, and
wherein determining the time difference (WWT) comprises
determining the time difference (WWT) for the plurality of successive heart periods as an average value based on the respective time differences determined for the plurality of heart periods.

40. The apparatus according to claim 38, wherein the first and second Gaussian functions have a respective maximum amplitude, the maximum amplitude of the first Gaussian function being greater than or equal to the maximum amplitude of the second Gaussian function.

41. The apparatus according to claim 38, wherein the first and second Gaussian functions have respective first and second standard deviations (σ₁, σ₂), the first and second standard deviations (σ₁, σ₂) being equal to each other.

42. The apparatus according to claim 37, wherein approximating the systolic component comprises:
fitting the first and second Gaussian functions to the systolic component using
$$F ( a , b , c , d , f ) = {\sum }_{i = 1}^{N} {( {S}_{i} - ( a \cdot {e}^{- \frac{1}{2} {( \frac{t - b}{c} )}^{2}} + d \cdot {e}^{- \frac{1}{2} {( \frac{t - f}{c} )}^{2}} ) )}^{2} {=}^{!} min$$
with a, b, c, and d being determined using non-linear optimization or curve-fitting.

43. The apparatus according to claim 38, wherein the regression model comprises a regression function
ƒ(SIₐ,g)=BP_{sys},
where SIₐ is an adjusted stiffness index (SIₐ), g is the gender of the human subject, and BP_{sys} is the blood pressure; and wherein determining the blood pressure value comprises determining the blood pressure value based on the regression function, wherein the regression function comprises a linear function of the type
ƒ(x)=ax+b,
wherein a ranges from 1 to 20 mmHg/(m/s) and b ranges from 0 to 80 mmHg.

44. The apparatus according to claim 38, wherein determining the adjusted stiffness index (SIₐ) is based on an adjustment function
ƒ(SIₚ)=SIₐ,
where SIₚ is the preliminary stiffness index and SIₐ is the adjusted stiffness index (SIₐ), wherein the adjustment function is a linear function of the type
ƒ(x)=cx+d,
where c and d are adjustment factors determined based on a plurality of value pairs comprising an age value and an associated stiffness index value.

45. The apparatus according to claim 38, wherein determining the systolic component comprises:
determining a respective global maximum of the respective heart period;
determining a second order derivative of the respective heart period;
determining a maximum value of the second order derivative located at least at a predetermined time difference from the global maximum; and
defining the systolic component as a portion of the heart period between a start of the heart period and the maximum value.

46. The apparatus according to claim 38, wherein determining the preliminary stiffness index (SIₚ) is based on a function
$${SI}_{p} = \frac{h}{WWT} ,$$
where h is the height of the human subject, WWT is the time difference, and SIₚ is the preliminary stiffness index (SIₚ).

47. The apparatus according to claim 31, wherein determining the at least one correlation value is based on the heart rate variability and further comprises:
generating, based on a plurality of heart rate variability values, a frequency distribution indicative of the distribution of the plurality of heart rate variability values in a time domain;
determining a plurality of expected values;
determining an entropy value indicative of a plurality of expected values, the entropy value being indicative of the medical condition of the human subject.

48. The apparatus according to claim 47, wherein the frequency distribution indicative of the distribution of the plurality of heart rate variability values comprises a histogram.

49. The apparatus according to claim 31, wherein the portion of the pulse wave data indicative of a plurality of heart periods covers a period of between 2 minutes and 5 minutes, and
wherein determining the variabilities of the blood pressure, respiratory rate, and the heart rate variability, is based on substantially all heart beats comprised in the pulse wave data.

50. The apparatus according to claim 31, wherein determining the at least one correlation value is based on the heart rate variability and the respiration rate variability and comprises detecting a correspondence between the heart rate variability and the respiration rate variability.
